(19) United States
(12) Patent Application Publication
(10) Pub. No.: US 2009/0022682 A1
(43) Pub. Date: Jan. 22, 2009

(54) FRAGRANCE DELIVERY SYSTEM

(75) Inventor: Michael Licciardello, Spartanburg, SC (US)

Correspondence Address: DORITY & MANNING, P.A., POST OFFICE BOX 1449, GREENVILLE, SC, 29602-1449, US

(73) Assignee: Grace Management Group, Spartanburg, SC (US)

(21) Appl. No.: 12/167,317

(22) Filed: Jul. 3, 2008

Related U.S. Application Data
(60) Provisional application No. 60/961,422, filed on Jul. 20, 2007.

(51) Int. Cl. A61L 9/04; A61L 9/12; A61L 9/01

(52) U.S. Cl. 424/76.21; 422/123

(57) ABSTRACT

Disclosed are apparatuses and methodologies for respectively delivering fragrances to desired environments. Formulations including volatile methylsiloxane fluid provide a clear, VOC-exempt, low surface tension fluid that will evaporate completely at room temperature. Because methylsiloxane fluid is itself an odorless fluid, it does not obscure or alter combined fragrances being otherwise delivered.

### PRIORITY CLAIM

This application claims the benefit of previously filed U.S. Provisional Patent Application entitled “FRAGRANCE DELIVERY SYSTEM,” assigned U.S. Ser. No. 60/961,422,filed Jul. 20, 2007, and which is hereby incorporated herein by reference in its entirety for all purposes.

### FIELD OF THE INVENTION

The present subject matter relates to apparatuses and methodologies for delivering fragrances within an area. More specifically, the present subject matter relates to volatile formulations used as a volatile organic compound (VOC) exempt carrier for fragrances.

### BACKGROUND OF THE INVENTION

Fragrance delivery systems and methodologies in various forms have been in use for many years. Such systems include candles, heated oils, atomizers, and diffusers devices including wick-type diffusers as well as other fragrance transport mechanisms. These devices may be used in a number of applications ranging from aromatherapy to environments odor control.

Individual known fragrance delivery systems may each have their own respective drawbacks, or overlapping or related drawbacks. Candles, for example, while effective to deliver fragrances within an environment and while often having various esthetically pleasing qualities, employ a flame which may be inappropriate in certain environments based on particularized fire risk, or other factors. Heated oils may also require either of a flame or an electric heating element which may pose similar drawbacks or others in particular circumstances.

Atomizer type devices may generally avoid various flame-related drawbacks associated with candles and heated oils, but such devices may require either of automated spray mechanisms (in turn requiring electrically operated pumps) or periodic manual operations that may be inconvenient or undesirable in certain environments.

Diffuser type delivery systems avoid many of the noted drawbacks as they generally do not require heat, flame, or external energy to affect fragrance deliver, but these too may be ineffective if inappropriate transport mechanisms in the form of ineffective wicking elements and/or combined fragrance delivery volatile mixtures are used.

One aspect of diffuser systems is that the Environmental Protection Agency (EPA) issues regulations regarding atmospheric emissions. More particularly, the EPA has a definition of so-called “volatile organic compounds” (VOC) for purposes of preparing state implementation plans relative to attaining the national ambient air quality standards for ozone under title I of the Clean Air Act. As adjunct to such defined compounds, a particular list of compounds is excluded from the definition of VOC on the basis that they make negligible contribution to any tropospheric ozone formation. In other words, they are considered to be VOC exempt.

While various VOC's may have a variety of potential uses including as heat-transfer fluid or substitutes for ozone depleting substances and substances with high global warming potentials (such as hydroflurocarbons, perfluorocarbons, and perfluoropolyethers), states regulate VOC emissions as precursors to ozone formation per the above-referenced national ambient air quality standards. The desire for such regulation is based in part on the fact that tropospheric ozone (commonly known as smog) occurs when VOC and nitrogen oxides (NOX) react in the atmosphere. Because of the harmful health effects of ozone, the EPA and state governments variously limit the amount of VOC and NOX that can be released into the atmosphere. Generally speaking, the VOC's are those particular compounds of carbon (excluding certain ones) which form ozone through atmospheric photochemical reactions. Compounds of carbon (also known as organic compounds) have different levels of reactivity. In other words, they do not react at the same speed or do not form ozone to the same extent.

It has been the EPA's policy that organic compounds with a negligible level of reactivity (in the process of forming ozone) need not be regulated to reduce ozone. The EPA has used a determination of whether a given organic compound has “negligible” reactivity by comparing the compound's reactivity to the reactivity of ethane. The EPA lists such compounds in its regulations as exclusions to its definition of VOC. The chemicals on such list are often called “negligibly reactive” organic compounds. In recent times, the EPA has used the reactivity of ethane as the threshold of negligible reactivity. Compounds that are less reactive than or equally reactive to ethane are potentially deemed to be negligibly reactive, while compounds that are more reactive than ethane continue to be considered reactive VOCs and subject to control requirements.

Various compositions which include fragrance materials are known in the patent literature, including U.S. Pat. No. 5,449,512 to Simmons and entitled “Anhydrous After Shave Lotions,” and U.S. Pat. No. 5,160,494 to Krzysik et al. and entitled “Alkylmethylsilox Containing Perfume Compositions.”

While various implementations of fragrance delivery systems have been developed, no design has emerged that generally encompasses all of the desired characteristics as hereafter presented in accordance with the subject technology.

### SUMMARY OF THE INVENTION

In view of the recognized features encountered in the prior art and addressed by the present subject matter, improved methodologies and corresponding apparatuses for delivering fragrances to environments have been developed.

In an exemplary configuration, a reed diffuser employing selected oil formulations has been developed.

In one of its simpler forms, diffuser oil formulations have been developed using volatile organic content (VOC) exempt carriers for fragrances.

Another positive aspect of such present type of device is that the VOC-exempt carriers are odorless and do not alter or obscure combined fragrances.

In accordance with aspects of certain embodiments of the present subject matter, methodologies are provided to effectively deliver fragrance to an environment over an extended period.

One present exemplary embodiment relates to a fragrance delivery system, comprising a container configured to contain a fluid; a fluid composition, and at least one wicking element. Preferably, such exemplary fluid composition may comprise 1-50% fragrance, 1-99% a volatile organic compound (VOC), and 1-99% solvent carrier. Further preferably for such exemplary embodiment, such VOC has a reactivity with nitrogen oxides less than or equal to that of ethane.

Another present exemplary embodiment relates more directly to a fragrance delivery fluid formulation. Such exemplary formulation may comprise 1-50% fragrance; 1-99% volatile organic compound (VOC) having a reactivity with nitrogen oxides less than or equal to that of ethane; and 1-99% solvent carrier. In such exemplary embodiment, preferably such VOC may comprise about twice the amount of such fragrance.

Additional details as referenced herein may be used in further alternative present combinations for providing further exemplary embodiments of a present fragrance delivery system or fragrance delivery fluid formulation.

Still further, it is to be understood that the present subject matter equally relates to associated methodology. In present exemplary methodology, one present exemplary methodology provided is for a fragrance delivery system. Such exemplary methodology may comprise providing a container configured to contain a diffuser oil; formulating such diffuser oil as a composition comprising 1-50% fragrance, 1-99% solvent carrier, and 1-99% a volatile organic compound (VOC) having a reactivity with nitrogen oxides less than or equal to that of ethane; and providing at least one wicking element comprising at least one reed.

Still further alternatives of such present exemplary embodiment may variously include additional features and/or steps, such as further disclosed herein.

Additional objects and advantages of the present subject matter are set forth in, or will be apparent to, those of ordinary skill in the art from the detailed description herein. Also, it should be further appreciated that modifications and variations to the specifically illustrated, referred and discussed features, elements, and steps hereof may be practiced in various embodiments and uses of the present subject matter without departing from the spirit and scope of the subject matter. Variations may include, but are not limited to, substitution of equivalent means, features, or steps for those illustrated, referenced, or discussed, and the functional, operational, or positional reversal of various parts, features, steps, or the like.

Still further, it is to be understood that different embodiments, as well as different presently preferred embodiments, of the present subject matter may include various combinations or configurations of presently disclosed features, steps, or elements, or their equivalents (including combinations of features, parts, or steps or configurations thereof not expressly shown in the figures or stated in the detailed description of such figures). Additional embodiments of the present subject matter, not necessarily expressed in the summarized section, may include and incorporate various combinations of aspects of features, components, or steps referenced in the summarized objects above, and/or other features, components, or steps as otherwise discussed in this application. Those of ordinary skill in the art will better appreciate the features and aspects of such embodiments, and others, upon review of the remainder of the specification.

### BRIEF DESCRIPTION OF THE DRAWINGS

A full and enabling disclosure of the present subject matter, including the best mode thereof, directed to one of ordinary skill in the art, is set forth in the specification, which makes reference to the appended figures, in which:

FIG. 1 illustrates a present exemplary reed diffuser configuration; and

FIGS. 2a and 2b illustrate a reed and a cross-section thereof as may be employed with the present subject matter.

Repeat use of reference characters throughout the present specification and appended drawings is intended to represent same or analogous features, elements, or steps of the present subject matter.

### DETAILED DESCRIPTION OF THE PREFERRED EMBODIMENTS

As referenced in the Summary of the Invention section, the present subject matter is particularly concerned with improved methodologies and corresponding apparatuses for delivering fragrances to environments.

There are several particular aspects to fragrance delivery systems as herein described in accordance with the present technology that contribute to commercial as well as functional success for such corresponding products. For example, more generally speaking, it is significant for users that any volatile components used be VOC-exempt relative to EPA requirements and regulations, as referenced above. Further, it is desirable that such fluids themselves be odorless so as not to obscure or add to any combined fragrance. Additionally, room temperature evaporation rates should preferably be such that any corresponding or resulting product will be effective for an extended period of time while maintaining sufficient fragrance transport into a surrounding environment. It is generally preferred that such fragrance transport formulations should be effective for relative duration of time, such as, for example, two to three months.

Applicants have found that methylsiloxane fluids, when presented and used per the present technology, demonstrate properties that contribute toward such aspects. One example of a particular such methylsiloxane fluid is produced by Dow Corning® and commercially available as Dow Corning® 244 Fluid (Dow 244). Dow 244 is a volatile methylsiloxane fluid that is clear, odorless, VOC-exempt, has low toxicity and low surface tension fluid, and evaporates completely at room temperature. Other methylsiloxane fluids suitable for use with the present technology include, but are not limited to, products offered by Dow Coming including: Dow Coming 344 Fluid, Dow Coming 345 Fluid, and Dow Coming OS-10, OS-20 and OS-30 Fluids.

Other materials commonly used as fragrance transport mechanism generally include ethanol, methoxy methylbutanol, dipropylene glycol methyl ether (available as Dow® DPM) and Exxon's IsoPar H series. None of these materials meet current VOC requirements except for ethanol, the characteristics of which are currently used as the limit of meeting VOC requirements. Some of these materials, especially methoxy methylbutanol, are particularly troublesome when used as fragrance carriers.

In accordance with the present technology, commercial as well as safety advantages are obtained through the use of present formulations. From a safety standpoint, present formulations meet National Fire Protection Association (NFPA) class 3A standards. That is the formulation flash point is greater than 140° F. but less than 200° F. so that while the product is combustible, it is non-flammable under present standards. Obtainment of such classification has significant advantages to all aspects of commercial storage (warehousing), transportation and retailing as such classification positively affects insurance and safety ratings for those individuals and companies involved with these aspects of such products.

Another significant aspect to present formulation is significantly reduced impact on top notes associated with multiple fragrances, most especially citrus fragrances. As those of ordinary skill in the art will appreciate, heat has significant adverse impact on fragrance top notes. Present formulations and deliver systems avoid degradation of fragrance top notes yet achieve diffusion comparable to that achieved with heated products including candles.

Selected combinations of aspects of the disclosed technology correspond to a plurality of different embodiments of the present subject matter. It should be noted that each of the exemplary embodiments presented and discussed herein should not insinuate limitations of the present subject matter. Features or steps illustrated or described as part of one embodiment may be used in combination with aspects of another embodiment to yield yet further embodiments. Additionally, certain features may be interchanged with similar devices or features not expressly mentioned which perform the same or similar function.

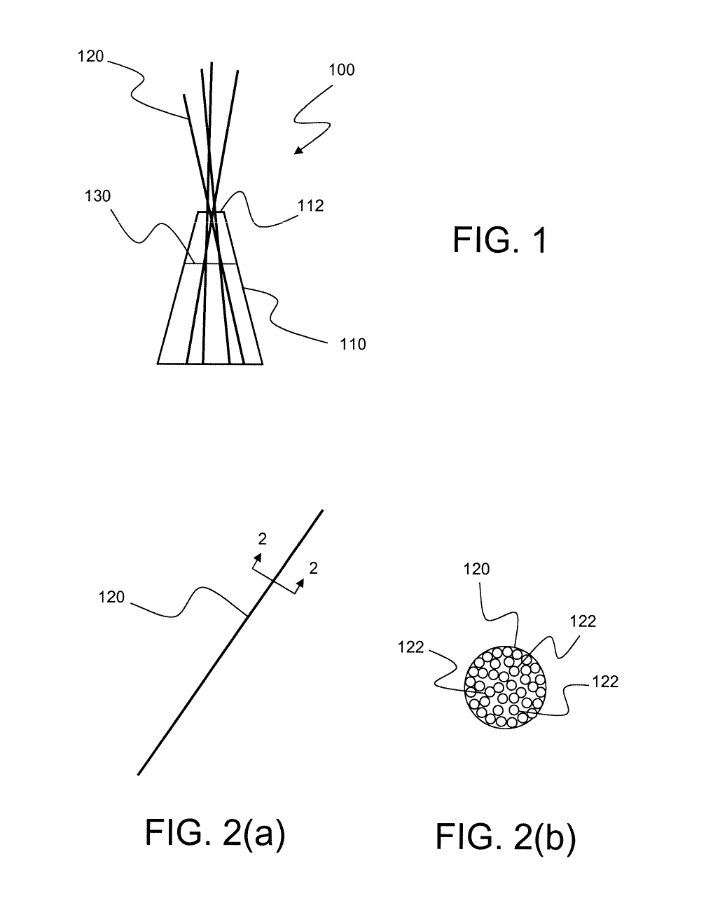

Reference will now be made in detail to presently preferred exemplary embodiments of the subject fragrance delivery system. Referring now to the drawings, FIG. 1 illustrates an exemplary reed diffuser configuration generally 100 as may be employed as a fragrance delivery system in accordance with the present technology. As illustrated in such FIG. 1, fragrance delivery system 100 may correspond to an exemplary container 110 in which are placed a number or plurality of reeds 120 (the number of which forms no particular aspect or limitation of the present subject matter). Container 110 may be configured to have a relatively small opening 112 and may be partially filled with a diffuser oil composition 130, as will be more fully described later.

It should be readily apparent to those of ordinary skill in the art that the exact shape of container 110 may be based generally on esthetics and suitability for a particular environment in which the inventive concept will be carried out. Thus, the exact shape of container 110 is not a specific limitation of the present subject matter. It will be appreciated, however, that a preferred configuration of container 100 per the present subject matter will generally include a reduced size opening 112.

With reference now to FIGS. 2(a) and 2(b), there is illustrated in FIG. 2(a) an individual reed 120 as may be employed with the present subject matter. There is illustrated in FIG. 2(b) an enlarged cross-section of exemplary reed 120 taken at section line 2-2 of FIG. 2(a), and generally illustrating the cell configuration thereof. Reed 120 may in the present example be about 3 mm in diameter, although such diameter is not a specific limitation of the present subject matter.

As seen in FIG. 2(b), reed 120 corresponds to a number of cellular structures generally 122 that extend the length of reed 120. Such cellular structures 122 may generally be thought of as a bundle of very small straws, which function by capillary action to wick fluid 130 up through the length of reed 120 such that an associated fragrance is carried by air currents throughout an area or environment within which such exemplary fragrance delivery system 100 is used.

While those of ordinary skill in the art will appreciate the general concepts of reed diffusers, the present technology provides relatively improved fragrance delivery achieved through present exemplary formulation of diffuser oil composition 130 in such diffusers. More specifically, the present technology makes advantageous use of volatile methylsiloxane fluids as a fragrance transport mechanism. Such fluids are particularly useful because they are odorless, volatile organic content (VOC) exempt, low in toxicity, low in surface tension, and highly effective for complete evaporation at room temperature. As a result, a fragrance can be dissolved into such fluids resulting in a desired product having similar advantageous properties.

An exemplary formulation in accordance with the present technology may correspond to fragrance, 1-50%; methylsiloxane fluid, 1-99%; and a solvent/carrier, 1-99%. Generally, the methylsiloxane fluid will be employed preferably at levels about twice that of the fragrance. The solvent/carrier is employed not only to address costing aspects but also to address desired solubility and stability aspects of the present technology. Suitable solvent/carriers may be selected from materials including, but not limited to, odorless hydrocarbon such as, but not limited to, Exxon's Iso Par Series; terpenes of essential oils such as, but not limited to, orange terpenes; and glycol ether such as, but not limited to, Dow's DPM.

### EXAMPLE 1

### In One Given Example, a Rose Reed Diffuser was Constructed and Tested as Follows

Formulation #1

| | |
|---|---|
| Rose Fragrance | 15% |
| IsoPar M | 50% |
| Orange Terpenes | 5% |
| Methylsiloxane fluid (Dow 244) | 30% |

Such Formulation #1 was blended together to yield 170 grams of Reed Diffuser Oil, which was then placed in an 8 oz. glass bottle. Ten 12-inch long reeds were placed in the bottle. The gross weight of such container and arrangement was 333.9 grams. The evaporation rate was monitored by weight loss over a period of time and resulting data collected as illustrated in Table 1.

TABLE 1

| | Weight of Container Minus | | Percentage of |
|---|---|---|---|
| # of Days | the Net Weight of Container | Gram Loss | Gram Loss |
| 7 | 136.1 | 33.9 | 19.9% |
| 24 | 86.1 | 83.9 | 49.4% |
| 34 | 68.9 | 101.1 | 59.5% |
| 85 | 0 | 170 | 100.0% |

### EXAMPLE 2

### In a Further Example, an Orange and Honey Reed Diffuser was Constructed and Tested as Follows

Formulation #2

| | |
|---|---|
| Orange and Honey Fragrance | 20% |
| IsoPar M | 50% |
| Methylsiloxane fluid (Dow 244) | 30% |

Such Formulation #2 was blended together to yield 170 grams of Reed Diffuser Oil, which was then placed in an 8 oz. glass bottle. Ten 12-inch reeds were placed in the bottle. The gross weight of such Formulation #2 container was 335.9 grams. The evaporation rate was then monitored by weight loss over the time periods, as reflected in present Table 2.

TABLE 2

| | Weight of Container Minus | | Percentage of |
|---|---|---|---|
| # of Days | the Net Weight of Container | Gram Loss | Gram Loss |
| 7 | 132.1 | 37.9 | 22.3% |
| 24 | 83.9 | 86.1 | 50.6% |
| 34 | 64.7 | 105.3 | 61.9% |
| 85 | 0 | 170 | 100.0% |

### EXAMPLE 3

### A Further Present Exemplary Hospitality Reed Diffuser was Constructed and Tested as Follows

Formulation #3

| | |
|---|---|
| Hospitality Fragrance | 15% |
| IsoPar M | 50% |
| Glycol Ether DPM | 5% |
| Methylsiloxane fluid (Dow 244) | 30% |

Such present Formulation #3 was blended together to yield 170 grams of Reed Diffuser Oil, which was then placed in an 8 oz. glass bottle. Then, 10 12-inch reeds were placed in such bottle. The gross weight of such container was 335.0 grams. The evaporation rate was then monitored by weight loss over the time periods as presented in Table 3 hereinbelow.

TABLE 3

| | Weight of Container Minus | | Percentage of |
|---|---|---|---|
| # of Days | the Net Weight of Container | Gram Loss | Gram Loss |
| 5 | 147.3 | 22.7 | 13.4% |
| 22 | 100 | 70 | 41.2% |
| 32 | 77 | 93 | 54.7% |
| 83 | 0 | 170 | 100.0% |

### EXAMPLE 4

### A Further Example by Way of Comparison Background was Constructed as an English Ivy Reed Diffuser and Tested as Follows

Formulation #4

| | |
|---|---|
| English Ivy Fragrance | 15% |
| IsoPar M | 80% |
| Orange Terpenes | 5% |

Such Formulation #4 was blended together to yield 170 grams of Reed Diffuser Oil, which was then placed in an 8 oz. glass bottle. Ten 12-inch reeds were placed in the bottle. The gross weight of such container was 333.4 grams. The evaporation rate was then monitored by weight loss over the below-referenced time periods, presented as the results of Table 4.

TABLE 4

| | Weight of Container Minus | | Percentage of |
|---|---|---|---|
| # of Days | the Net Weight of Container | Gram Loss | Gram Loss |
| 8 | 163.8 | 6.2 | 3.6% |
| 14 | 160.8 | 9.2 | 5.4% |
| 35 | 150.3 | 19.7 | 11.6% |
| 67 | 132.5 | 37.5 | 22.1% |

As may be observed from a comparison of the data from Tables 1-4, in those instances (exemplary Formulation Nos. 1 through 3) where the methylsiloxane fluid (Dow 244) formed 30% of the formulation, the observed percentage of gram loss reached 100% in 83-85 days. In the case of exemplary Formulation #4, where no methylsiloxane fluid was included in the formulation, percentage of gram loss reached only 22. 1% after 67 days. Such significantly lower loss demonstrates that the fragrance was not being transported to the extent of that of present Formulation Nos. 1 through 3. Thus, while exemplary Formulation #4 illustrates a formulation that might well last four times as long (that is, take four times as long to reach 100% gram loss), such formulation may not be considered as appropriate or sufficiently effective for the present purposes of fragrance delivery.

It should be appreciated by those of ordinary skill in the art that while the present subject matter has been described as used in a reed diffuser configuration, such is not a limitation of the present subject matter. Generally, the exemplary formulations may be used in other type diffusers, including other wicking type devices as well as other devices including, for example, formula permeated pads that may be exposed to ambient air flow for fragrance delivery.

While the present subject matter has been described in detail with respect to specific embodiments thereof, it will be appreciated that those skilled in the art, upon attaining an understanding of the foregoing, may readily produce alterations to, variations of, and equivalents to such embodiments. Accordingly, the scope of the present disclosure is by way of example rather than by way of limitation, and the subject disclosure does not preclude inclusion of such modifications, variations, and/or additions to the present subject matter as would be readily apparent to one of ordinary skill in the art.

What is claimed is:

1. A fragrance delivery system, comprising:
a container configured to contain a fluid;
a fluid composition comprising 1-50% fragrance, 1-99% a volatile organic compound (VOC), and 1-99% solvent carrier; and
at least one wicking element;
wherein said VOC has a reactivity with nitrogen oxides less than or equal to that of ethane.

2. A fragrance delivery system as in claim 1, wherein said fluid composition has a flash point greater than 140° F. but less than 200° F.

3. A fragrance delivery system as in claim 1, wherein said VOC is odorless.

4. A fragrance delivery system as in claim 1, wherein said VOC comprises methylsiloxane.

5. A fragrance delivery system as in claim 1, wherein said VOC comprises about twice the amount of said fragrance.

6. A fragrance delivery system as in claim 1, wherein said VOC comprises about 30% of said fluid composition.

7. A fragrance delivery system as in claim 1, wherein said solvent carrier comprises an odorless hydrocarbon.

8. A fragrance delivery system as in claim 7, where said solvent carrier comprises an isoparaffin.

9. A fragrance delivery system as in claim 1, wherein said solvent carrier comprises about 50% of said fluid composition.

10. A fragrance delivery system as in claim 1, wherein said at least one wicking element comprises a reed.

11. A fragrance delivery system as in claim 10, wherein said at least one wicking element comprises a cellular structure extending the length of said wicking element, whereby said fluid composition may be wicked thereby per capillary action.

12. A fragrance delivery system as in claim 10, wherein:
said at least one wicking element has a diameter of about 3 mm; and
said container is configured so as to have a generally reduced size opening through which said at least one wicking element is received.

13. A fragrance delivery fluid formulation, comprising:
1-50% fragrance;
1-99% volatile organic compound (VOC) having a reactivity with nitrogen oxides less than or equal to that of ethane; and
1-99% solvent carrier;
wherein said VOC comprises about twice the amount of said fragrance.

14. A fragrance delivery fluid formulation as in claim 13, wherein the flash point of said formulation is greater than 140° F. but less than 200° F.

15. A fragrance delivery fluid formulation as in claim 13, wherein said VOC is odorless.

16. A fragrance delivery fluid formulation as in claim 13, wherein said VOC comprises methylsiloxane.

17. A fragrance delivery fluid formulation as in claim 13, wherein said solvent carrier is an odorless hydrocarbon.

18. A fragrance delivery fluid formulation as in claim 13, wherein said solvent carrier comprises an isoparaffin.

19. A fragrance delivery fluid formulation as in claim 13, wherein said solvent carrier comprises about 50% of said fluid formulation.

20. A fragrance delivery composition as in claim 13, wherein said VOC comprises about 30% of said fluid formulation.

21. A methodology for a fragrance delivery system, comprising:
providing a container configured to contain a diffuser oil;
formulating such diffuser oil as a composition comprising 1-50% fragrance, 1-99% solvent carrier, and 1-99% a volatile organic compound (VOC) having a reactivity with nitrogen oxides less than or equal to that of ethane; and
providing at least one wicking element comprising at least one reed.

22. A methodology as in claim 21, further comprising formulating the diffuser oil so as to have a flash point greater than 140° F. but less than 200° F.

23. A methodology as in claim 21, further comprising formulating the diffuser oil so that the VOC comprises about twice the amount of the fragrance.

24. A methodology as in claim 21, further comprising formulating the diffuser oil so that the solvent carrier comprises about 50% of the fluid composition.

25. A methodology as in claim 21, wherein:
the wicking element comprises a plurality of reeds, respectively each having a cellular structure having a diameter of about 3mm and extending the length of the wicking element, whereby the diffuser oil may be wicked thereby per capillary action; and
wherein the container is configured so as to have a generally reduced size opening through which the wicking element is received.
